(19) United States
(12) Patent Application Publication
(10) Pub. No.: US 2026/0330228 A1
(43) Pub. Date: Oct. 22, 2026

(54) DEVELOPMENT PROCESS FOR FORMULATING COMPOSITE MASSES AND PREDICTING PROPERTIES OF CAST WORKPIECES PRODUCED THEREFROM

(71) Applicant: Gebrüder Dorfner GmbH & Co. KG, Hirschau (DE)

(72) Inventors: Vanessa COLELLI, Amberg (DE); Marco SÜß, Neunburg vorm Wald (DE)

(73) Assignee: Gebrüder Dorfner GmbH & Co. KG, Hirschau (DE)

(21) Appl. No.: 19/538,974

(22) Filed: Feb. 12, 2026

(30) Foreign Application Priority Data
Feb. 17, 2025 (DE) 10 2025 105 870.9

(51) Int. Cl. G06N 3/084; G06F 16/28; G06N 3/045; G06N 3/08; G06N 20/00

(52) U.S. Cl. CPC G06N 3/084; G06F 16/285; G06N 3/045; G06N 3/08; G06N 20/00

(57) ABSTRACT

Method for the predictive generation of a first composition dataset (ZD₁), comprising data for formulation of a first filler composition (FM₁) and/or a first composite mass (KM₁) for production of a first cast workpiece (GWS₁). The method comprises the steps: capturing a second composition dataset (ZD₂) relating to a second filler composition (FM₂) and/or second composite mass (KM₂) and/or second cast workpiece (GWS₂), capturing at least one further composition dataset (ZD₃ …ZDₙ) relating to at least one further filler composition (FM₂) and/or further composite mass (KM₃ …KMₙ) and/or further cast workpiece (GWS₃ …GWSₙ), where the composition datasets comprise at least one analysis variable (AGₓ) and a proportion value (NAₓ) of a component (Aₓ), the analysis variable being characteristic of at least one property (Eₓ) of the filler composition (FM₂ …FMₙ) and/or composite mass (KM₂, … KMₙ) and/or cast workpiece (GWS₂ …GWSₙ) characterized by the respective composition dataset (ZD₂ …ZDₙ).

### CROSS-REFERENCE TO RELATED APPLICATION

The present application claims priority to German Patent Application No. 10-2025-105870.9, filed Feb. 17, 2025, the entire contents of which are incorporated by reference herein.

### BACKGROUND OF THE INVENTION

### Description

The present invention relates to a method for generating a first composition dataset comprising data for formulating a filler and/or a first composite mass for producing a first cast workpiece. Furthermore, the invention relates to a filler and/or composite mass formulation determination device, for the determination, in particular for predictive determination of at least one property of a first filler, a first composite mass, and/or a first cast workpiece. Furthermore, the invention relates to a method for generating a (trained) machine learning filler and/or composite mass formulation model for determining at least one first property of a first filler solution and/or a first composite mass, and/or a first cast workpiece that can be produced from it based on the use of composition datasets and using this model to determine or identify at least one analysis variable characteristic of at least one property of a first filler solution and/or composite mass and/or a first cast workpiece.

The state of the art includes a wide variety of different fillers and composite masses, as well as cast workpieces made from them. These are adapted to the respective tasks and application scenarios. Well-known applications for composite masses and cast workpieces made from them include kitchen sinks, sanitary products such as washbasin, shower trays and enclosures, bathtubs, toilets, and bidets. They are also used in construction, furniture manufacturing, and automotive engineering. For example, cast workpiece from composite mass are used as countertops, flooring, or wall coverings, electrical insulation profiles, furniture parts, writing instruments, urns, household appliances, door interior panels, and hat racks.

A large number of different composite masses are already known from the prior art. These usually consist of particulate inorganic fillers, such as minerals (e.g., quartz sand), that are embedded in an organic binder (e.g., polyester or acrylic) to form the composite material.

In addition to these composite masses with mineral fillers, there are also composite mass substances are known in which synthetic fillers (e.g., ATH) are embedded in organic binders (e.g., known as "solid surface material"). It is also known that mixtures of different minerals can be used as fillers in a composite mass.

Polymers containing fillers have been produced and used on an industrial scale for many decades. For the composite mass described above, polymers such as thermoplastics, thermosets or elastomers, or mixtures of these three classes in various proportions, are used.

The polymers or composite masses containing these polymers may comprise additives to modify certain properties in a composition, e.g., rheology, elasticity, fracture behavior and weathering resistance. The additives may be organic, inorganic or mixtures thereof.

Compositions for composite masses are also known in which the filler fraction contains additives to influence certain properties of the fillers themselves or their effect in the polymer, e.g., rheology, elasticity, fracture behavior, weathering resistance, incorporation, processing, durability, color, and scratch and abrasion resistance (according to DIN EN 13310), etc. Coloring may be achieved using dyes or pigments. The additives may be organic, inorganic, or mixtures of organic and inorganic additives. They can be present as loose mixtures with other fillers or may be physically, mechanically, or chemically bound to the filler. Physical or chemical bonding to the polymer is also possible.

The prior art includes, for example, acrylic-based molded parts containing fillers (e.g., quartz, ATH, etc.) which are manufactured into a molded part using a thermoforming process and used, for example, as kitchen sinks, especially washbasins, shower trays, or bathtubs. In addition to quartz and ATH, other fillers and additives may also be used.

Furthermore, the so-called gelcoat process is known from the prior art. For this purpose, a negative mold is sprayed with a first resin layer, which may also contain filler, and which forms the visible side of the subsequent molded part. This first layer is backfilled with a second layer of resin containing filler. Both polymer layers may contain the above-mentioned fillers and additives.

The prior art also includes the so-called mineral cast workpiece process, also known as the solid surface process. Here, filler-containing polymers are injected into a mold using pressure feed, where they cure partly catalytically and partly thermally. CaCO₃ and ATH are mainly used as fillers, along with additives and, if necessary, coloring components.

In addition, composite materials, depending on their properties and composition, can be used in injection molding or 3D printing. However, such applications have to date been limited to niche products.

Most of the known processes have in common that the particulate components are mixed with a) a resin syrup made of PMMA and MMA and, under pressure and at elevated temperatures in molded parts or b) cast with polyester in cast workpiece machines to form molded parts and cured with the aid of hardening additives. The polyester resin used in b) may optionally also contain a certain amount of methyl methacrylate (MMA). The syrup mentioned in a) may also contain isobornyl methacrylate (I-BOMA) and cyclohexyl methacrylate (CHMA) as well as ethylene glycol dimethyl acrylate (EGDMA), 1,6-hexanediol dimethacrylate (1,6-HDDMA) and triethylene glycol dimethacrylate (TRGDMA), the latter two generally used as crosslinkers.

It is increasingly common for both polymers and fillers to be derived from renewable resources.

For example, organic fillers based on renewable resources are increasingly being used. These include nut shells or olive pits, fibers, pods, seeds, or other plant components, which are used, for example, as flour or as particles ground to a specific grain size. The fillers used can also be based on recycled materials. These can be mineral-based or contain organic components.

Examples of molding compounds with polymers or binders based on renewable resources are also known. Both polymers a) and b) can also be completely or based on recyclates or mixtures thereof.

Since this wide range of applications also places different demands on the respective composite mass or composite mass used to manufacture them, it is necessary to adapt the filler and composite mass for almost every application. A composite mass suitable for one application is usually not ideal for another application. The use of composite mass that are not optimally designed for a specific application is generally uneconomical, as they often contain costly raw materials in increased quantities that would not be necessary for the intended application, or at least not in such amounts.

Accordingly, it is technically advantageous and generally economically reasonable to adapt the fillers and composite mass to a specific application. However, this entails significant development costs in the current state of the art. The respective filler and composite mass must be adapted to the intended application, and applications must be developed and tested to ensure suitability. To date, a large number numerous of filler solutions and composite mass have been produced on a laboratory scale and their properties measured in laboratory tests, some of which were complex. Fillers and/or composite masses that are not suitable for use in a process for converting them into a workpiece due to their properties (e.g., due to their rheological behavior) had to be discarded at this stage of development.

However, testing the conversion of formulation candidates that are plausible in the laboratory into cast workpieces is even more time-consuming and involves generates additional waste. The conversion to cast workpieces is very time-consuming, particularly due to the time required for polymerization and/or curing of the binder. In addition, parallel testing of various composite mass candidates is often only possible to a limited extent due to the number of suitable cast workpiece molds available. In particular, testing for suitability in forming complex geometric shapes (e.g., with tight radii of curvature) is therefore only possible to a limited extent. Finally, the material composition, wall thickness, and consequently the costs for corresponding process tests are very high, since a stable process must first be established for each test variant, and the cast workpieces produced usually have to be disposed of. The negative aspects in terms of sustainability constitute an additional factor.

In the past, studies were therefore often conducted that only examined the effects of changing individual ingredients or their proportions on various parameters of the composite mass or the cast workpiece. However, for the reasons mentioned above, determining the effect of individual components on the properties of the composite mass or the cast workpiece remains time-consuming and costly.

Furthermore, such investigations are not very reliable, as these series of tests may not accurately reflect synergistic or adverse effects between different components of a composite mass or the cast workpiece. If such effects were also to be taken into account, an even significantly larger number of laboratory tests would be necessary, which would in most cases exceed the cost-benefit ratio. In addition, such an investigation would in most cases result in an enormous amount of data, the evaluation of which would also require considerable time.

Until now, attempts have been made to identify a few components of fillers, composite masses or cast workpieces and to determine their mutual synergistic or adverse effects within a composite mass or composite material. However, these investigations do not provide satisfactory results, since it is necessary to limit the number of components and, at the same time, a large volume of data is generated and must be evaluated.

### SUMMARY OF THE INVENTION

The present invention is therefore based on the task of overcoming the disadvantages known from the prior art and providing a method and a property determination device for determining formulations for a) filler mixtures for b) a composite mass and c) for production of a cast workpiece. This should enable a reliable, fast, and computationally efficient prediction at least one property of a filler solution and/or composite mass and/or a cast workpiece producible therefrom.

According to the invention, the object is achieved by the features defined in the independent claims. Preferred embodiments and further developments of the invention are specified in the dependent claims.

In a method according to the invention for generating a first composition dataset, in particular a predictive composition dataset, the composition dataset comprises data for formulating a first filler composition and/or a first composite mass for producing a first cast workpiece. This first composition dataset comprises:
information relating to a first component and at least one second component different from the first component,
a proportion of the first component and a proportion of the second component in the first filler composition and/or the first composite mass and/or the first cast workpiece, and
a proportion of the first component and a proportion of the second component in the first composite mass and/or the first cast workpiece, and
information on at least one property of the first composite mass and/or the first cast workpiece that can be characterized by a first analysis variable.

The method comprises the following steps:
a) acquisition of a second composition dataset relating to a second filler composition. The second composition dataset comprises at least one second analysis variable and a proportion value of a second component and, preferably, a plurality of proportion values of additional components.
b) recording at least one further composition dataset relating to at least one further filler composition and/or a further composite mass and/or a further cast workpiece. The at least one further composition dataset comprises at least one further analysis variable and a further proportion value of a further component and, preferably, a plurality of further proportion values of further components. In addition, the second and/or further analysis variable is characteristic of at least one property of the filler composition and/or composite mass and/or cast workpiece characterized by the respective composition dataset. Furthermore, in such a further composition dataset, the second and/or further proportion value is characteristic of a quantity fraction of the first and/or further components present in the filler composition and/or composite mass and/or cast workpiece characterized by the respective composition dataset.
c) computer-implemented determination of at least one value of the first composition dataset based on the second composition dataset and at least one further composition dataset. The determined value is selected from information from a group of information
   concerning the presence or absence of a first component and/or the at least one further component in the first filler composition and/or composite mass and/or the first cast workpiece,
   concerning a proportion of the first and/or further component in the first filler composition and/or the first composite mass and/or the first cast workpiece, and
   concerning at least one first property of the first filler composition and/or the first composite that can be characterized by the first analysis variable, mass and/or the first cast workpiece.

In this context, a cast workpiece should be understood to mean any workpiece that can be produced from a composite mass and a filler composition. It is irrelevant whether the cast workpiece is actually produced by casting the composite mass or by another process. Other conceivable processes include thermoforming, 3D printing, or injection molding.

Information on a (first) component of a filler composition and/or a composite mass and/or the cast workpiece within the scope of this invention should be understood to mean any information available on this component. This information may, for example, be encoded as a numerical value or a binary code that can be uniquely assigned to this information. However, the proportion of this component in the filler composition and/or the composite mass and/or the cast workpiece is preferably not included such information, since the value is preferably handled separately and stored in the composition dataset.

In the context of this invention, information relating to a first component of the first filler composition and/or the first composite mass and/or the first cast workpiece is often marked with the reference symbol "A₁". The letter "A" represents the substance or mixture of substances contained in the first filler composition and/or the first composite mass and/or the first cast workpiece. The index "₁" can provide information about a special characteristic or property of this substance or mixture of substances, as contained in the first filler composition and/or the first composite mass and/or the first cast workpiece.

For example, the index may contain a manufacturer, date of manufacture, batch number, purity, concentration, specific impurity, solvent, solvent content, particle size, particle size distribution, molar mass distribution, structure, degree of branching, melting point, glass transition temperature, or other properties of the substance or mixture of substances, including combinations of such properties. The reference symbol "A₁" is to be understood as meaning that substance "A" is used with the specific properties known for this first component. The reference symbol "A₂" accordingly stands for the same substance with the properties known for this second component. Some properties may be identical.

If components "A₁" and "A₂" differ significantly, the component may be represented by a different letter, for example "B," instead of the letter "A." This may be useful, for example, in the case of copolymers, which are composed of the same monomers but contain them in different proportions, different sequences, and/or with a different degree of cross-linking.

Conversely, the first component "A₁" and the second component "A₂" may not only be the same substance but may also be identical in all other properties. The only difference between such a first component "A₁" and this second component "A₂" in such a case is then that "A₁" is a component of the first filler composition and/or the first composite mass and/or the first cast workpiece, and "A₂" is a component of the second filler composition and/or the second composite mass and/or the second cast workpiece.

A proportion of a component in a filler composition and/or composite mass and/or a cast workpiece, unless otherwise specified, should be understood as information on the weight proportion of the respective component in a filler composition and/or composite mass and/or a cast workpiece. It should be noted that even if a cast workpiece is produced from a specific composite mass or filler composition, the proportion of the respective component in the composite mass and/or the cast workpiece may vary. Such a difference may result, for example, from changes in proportions during polymerization, evaporation of solvent, and/or the separation of reaction products (for example, gaseous) during the transformation into a cast workpiece. The reference symbol "N_{A2}" for a proportion indicates that proportion "N" refers to component "A₂" – i.e., the substance A as present in the second filler composition and/or the second composite mass and/or the second cast workpiece. Of course, it is possible that the proportions of various components in one or more filler compositions and/or composite masses and/or cast workpieces may be identical.

It is preferable that each component, in particular each individual component, in a composition dataset is assigned a proportion. Preferably, each proportion refers to a component, specified and/or specifiable, assigned to it in the filler composition, and/or composite mass and/or cast workpiece characterized by the respective composition dataset.

Information on at least one property of a filler composition and/or a composite mass and/or a cast workpiece characterized by an analysis variable should be understood as a value that, when analyzing this composite mass and/or this cast workpiece is measurable or predictable in terms of a specific property. For example, a property of a filler composition and/or a composite mass and/or a cast workpiece can be a color that can be characterized by a corresponding analysis variable, for example values of a frequency spectrum of the visible wavelength range. Further examples of possible analysis variables are viscosity, gloss, color distribution, roughness, sound absorption, density, density distribution, hardness, modulus of elasticity, ductility, impact strength (impact toughness), breaking strength, tear resistance, elasticity, bending stiffness, temperature resistance, temperature change resistance, melting point, softening temperature, decomposition temperature, viscosity, UV resistance, wear resistance, corrosion resistance (e.g. to water, humid air, oxygen), acid resistance or alkali resistance, combustibility, and (eco)toxicity.

The term "recording of a (second, third, or further) composition dataset" refers in particular to receiving a composition dataset transmitted via a communication device and/or retrieving the composition dataset of a storage device, in particular a non-volatile storage device (such as an external storage device, for example an external backend server), by a composite mass formulation determination device, in particular a processor-based device described in more detail below, so that preferably one or more composition datasets are recorded and provided for data processing.

In addition, according to a further embodiment, the term "recording of a (second, third, or further) composition dataset" may additionally or alternatively also be understood as determining and/or generating the composition dataset. This could be achieved by mathematical calculation, for example by interpolation and/or extrapolation of specified data values (for example, at least one or more similar filler compositions and/or composite masses and/or cast workpieces) and/or by experimental generation or experimental determination and/or experimental measurement (of values) of the composition dataset.

As explained above, a composition dataset contains data relating to analysis variable(s), proportion(s), and component(s). A value contained in the composition dataset preferably represents a unique and, in particular unambiguous, analysis variable, proportion, and component. The term "value" can be understood as a single entry in a data record and/or a single data point in a group of data. Such a value can be a number, a function, a letter, a sequence of letters, or combinations thereof.

In the following, no distinction is made between a value (e.g., specified as binary code) value that characterizes an analysis variable, a proportion value, or a component in a composition dataset and the actual value of the analysis variable (e.g., density 1.2 kg/l) or proportion value (e.g., 5 % by weight), or the actual value of a filler composition and/or composite mass and/or a cast workpiece (e.g., quartz). Unless explicitly stated otherwise, within the scope of this description of the invention, a disclosure relating to a value of a composition dataset that characterizes an analysis variable, a proportion value, or a component is also to be understood as a disclosure relating to the actual value of the analysis variable or proportion value, or the actual value in a filler composition and/or a composite mass and/or a cast workpiece. Similarly, a description of an actual value of the analysis variable or proportion value, or of the component actually contained in a filler composition and/or a composite mass and/or a cast workpiece, shall also be considered a disclosure of a value in a composition dataset which contains this analysis variable, proportion value, or component.

Analogously, within the scope of this description of the invention, any description made for a composition dataset shall also be deemed disclosed for the filler composition and/or the composite mass and/or the cast workpiece, which is characterized by this composition dataset, unless explicitly excluded. Conversely, however, disclosure given for a filler composition and/or a composite mass and/or a cast workpiece shall also be considered disclosure for a composition dataset characterizing this filler composition and/or this composite mass and/or this cast workpiece.

Similarly, within the scope of this description of the invention, any description relating to a property of a filler composition and/or a composite mass and/or a cast workpiece shall also be considered disclosed for a corresponding analysis variable, and conversely, since a clearly defined analysis variable contained in a composition dataset preferably characterizes a property of a filler composition and/or a composite mass and/or a cast workpiece.

By means of the method described above, it can preferably be enabled that a first composition dataset to be determined, which represents a suitable first filler composition and/or a formulation of a first composite mass, which (together or independently of each other) are suitable for producing a first cast workpiece. The use of the first filler composition and/or the first composite mass preferably results in a cast workpiece having properties that were previously specified as target parameters. Preferably, he filler composition and/or composite mass defined by the first composition dataset can be converted into a cast workpiece with these properties. It may be possible that the first filler composition determined in this way is first converted into a first composite mass (preferably defined by a separate composition dataset). Alternatively, only the composition of the first filler composition could be determined by the method described above, and this filler composition could then be used in a predefined (standard) formulation for a composite mass. Nevertheless, even with this variant, the use of the first filler composition determined by the method described above or defined by the composition dataset, preferably results, when processed into a cast workpiece, in a cast workpiece having the desired properties. Accordingly, the method described above may be single-stage or multi-stage.

Preferably, the computer-implemented determination of at least one value of the first composition dataset is carried out on the basis of a plurality of composition datasets that have been recorded for various filler compositions and/or composite masses and/or cast workpieces. In this variant of the method, it is thus possible to determine a value, preferably an analysis variable of the first composition dataset, and thereby also a property of the first filler composition and/or the first composite mass and/or the first cast workpiece, without the first filler composition and/or first composite mass and/or the first cast workpiece must actually be analyzed (physico-chemical) for this analysis variable and/or property. Rather, it is possible to determine this analysis variable and/or property (preferably predictively) by calculation (preferably computer-aided). The basis for these calculations is data from composition datasets that are, for example, stored in a database for this purpose.

Preferably, each composition dataset (preferably through the values of the proportions assigned to the respective components) is characteristic of a filler composition and/or a composite mass and/or a cast workpiece, so that, in particular, the different filler compositions and/or composite masses and/or the first cast workpieces are preferably distinguishable from one another (preferably unambiguously) on the basis of the respective composition datasets. However, it would also be conceivable that two (or more) filler compositions and/or composite and/or cast workpieces are so similar in their composition/formulation that their respective composition datasets are the same (essentially or, if applicable, within a (measurement) tolerance).

Preferably, the first filler composition and/or first composite mass and/or the first cast workpiece may contain a plurality of components, for example more than two, preferably more than three, preferably more than five, preferably more than eight, preferably more than ten, and particularly preferably more than 15 components in the first filler composition and/or the first composite mass and/or the first cast workpiece, or to the first filler composition and/or the first composite mass and/or the first cast workpiece. The components are understood to be, in particular, substances or mixtures that can be actively combined to obtain the first filler composition and/or the first composite mass and/or the first cast workpiece. Any impurities contained in one or more of the components remain in this consideration.

If any impurities contained therein are also considered as separate components, their number in the first composite mass and/or the first cast workpiece may also be significantly greater than 50 or even > 100, since the B components are often only used in technical purity and/or, for example, a purity of ≥ 99 percent by weight.

The data contained in the composition dataset or datasets relating to analysis variable(s), proportion value(s), and component(s) may each, preferably independently of one another, be determined by calculation or by (physico-chemical) analysis. Preferably, however, at least a predominant portion, preferably ≥ 75 %, more preferably ≥ 90 %, even more preferably ≥ 95 %, and most preferably ≥ 99 % of the data contained in the composition dataset or datasets is determined by (physico-chemical) analysis. A high proportion of data determined by (physico-chemical) analysis in the composition dataset or datasets has the advantage that such data is usually less error-prone than data determined by calculation, since possible errors in the data forming the basis of the calculation may be carried over or even amplified.

Preferably, the second composition dataset is obtained by an analysis, preferably a full analysis, of the second filler composition and/or the second composite mass and/or a second cast workpiece and/or the at least one further composition dataset is preferably obtained by analyzing, preferably fully analyzing, the at least one further composite mass. An analysis may be limited to determining one or more of the data contained in the composition dataset. As an alternative to analyzing all data in the dataset, a value or several data values, in particular one or more proportion variables, can also be taken from or calculated from a formulation or formulation instruction. In particular, it is therefore preferable that one or more analysis variables are determined by analyzing the respective property of the respective filler composition and/or the respective composite mass and/or the respective cast workpiece and/or the proportions of the first, second, and/or further components of a formulation or formulation instruction are taken from or calculated on the basis of this. This reduces the amount of analysis required.

Determination of a property of a filler composition and/or a composite mass and/or a cast workpiece based on data that is already known during the production of this filler composition and/or this composite mass and/or this cast workpiece (namely, in particular, the components used and their respective proportion values), and which may be rechecked during transport and/or storage and/or further conversion into a cast workpiece, involves significantly less effort compared to an experimental measurement of the properties of the filler composition and/or composite mass and/or cast workpiece.

A composition dataset preferably comprises pairs of data (hereinafter also referred to as descriptors), each of which is characteristic of a component and its proportion value in the filler composition and/or composite mass and/or cast workpiece characterized by this composition dataset. Furthermore, a composition dataset preferably comprises at least one value that is characteristic of an analysis variable. Preferably, however, a composition dataset contains a plurality of values characteristic of analysis variables, each value preferably being characteristic of (preferably exactly) one analysis variable.

In particular, it is preferred that a composition dataset (preferably each or at least nearly every composition dataset) comprises several of the above-mentioned pairs of data. It has been shown that a filler composition and/or a composite mass and/or a cast workpiece can be characterized with sufficient accuracy provided that at least two such pairs are defined. Such a small amount of data is particularly sufficient if one of the components characterized by such a pair of data is a mixture of several substances. However, a composition dataset preferably comprises several of the above-mentioned pairs of data, preferably at least 4, more preferably at least 6, even more preferably at least 8, and most preferably at least 10 of the above-mentioned pairs of data.

Preferably, a composition dataset comprises pairs of data that are characteristic of components and their proportion values, which are present in such a large proportion in the filler composition and/or the composite mass and/or the cast workpiece so that they have a significant influence on at least one property of the respective filler composition and/or the composite mass and/or the respective cast workpiece, in particular on one or the property represented by the analysis variable. Since a large proportion of the filler composition and/or the composite mass and/or the cast workpiece may be formed by a polymer and/or a filler, the proportion of one (or more) other component(s) may be very small, and the respective component may nevertheless have a noticeable influence on the overall filler composition and/or the composite mass and/or the cast workpiece. Accordingly, no lower limit on the proportion is preferably defined. However, it has been shown that proportions regarded as mere contamination may be negligible. Preferably, therefore, a composition dataset comprises pairs of data in which the respective proportion has a (weight) share of at least 1:1,000,000 (based on the total weight of all components in a filler composition and/or a composite mass and/or the cast workpiece), preferably at least 1:100,000, and most preferably 1:10,000.

By limiting the data to pairs of data relevant to at least one analysis variable in a composite dataset, the amount of data can be kept relatively small. For example, a limitation to ≤ 50, preferably ≤ 40, further preferably ≤ 30, more preferably ≤ 25, and most preferably ≤ 20 pairs of data per composition dataset.

It is also advantageous to limit the number of possible components. This is not only advantageous in terms of the size of the dataset to be handled, in particular, it also reduces the necessary computing power. It is therefore advantageous to have a number of permitted components ≤ 1000, preferably ≤ 750, more preferably ≤ 500, and more preferably ≤ 400 and most preferably ≤ 350. Preferably, those components are selected which are contained in commercially known filler compositions and/or composite masses and/or cast workpieces and/or which are known or at least suspected to have a significant influence on at least one analysis variable.

Preferably, the composition dataset (especially each one) should have at least one analysis variable. For example, it would be conceivable to use only the viscosity of the composite mass as the sole analysis variable. This information could be used, for example, to evaluate different filler compositions and/or composite masses for producing cast workpieces with particularly tight curve radii and/or complex geometries.

However, preferably each composition dataset comprises not only a single analysis variable but several analysis variables. It is common practice for filler compositions and/or composite masses and/or cast workpieces to be characterized by multiple data items that are relevant to users. These data are usually determined for a filler composition and/or composite mass and/or cast workpiece anyway, so that they are available and can be incorporated comparatively easily into a composition dataset. The specification of several analysis variables in a composition dataset is advantageous because it enables the determination not only of the (physico-chemical) property of the filler composition and/or composite mass and/or cast workpiece, but also of other properties. An expert or a computer system can thus immediately perform a plausibility check and exclude or reject determination results for which at least one property or analysis variable lies outside a permissible range. Preferably, each composition dataset comprises at least 3, ≥ 5 analysis variables, preferably ≥ 8, more preferably ≥ 12, even more preferably ≥ 15, particularly preferably ≥ 20, and most preferably ≥ 25 analysis variables.

Additionally or alternatively, each composition dataset preferably comprises ≤ 50, preferably ≤ 30, more preferably ≤ 25, and most preferably ≤ 20 different analysis variables. In principle, it would also be possible to use composition datasets with a larger number of different analysis variables; however, in view of the amount of data to be stored and the required computing power, the number of relevant analysis variables is preferably limited to the above-mentioned range. For most commercially available filler compositions and/or composite masses and/or cast workpieces, the product information also specifies only a significantly smaller number of relevant characteristic data for characterizing the product properties.

The aforementioned maximum numbers for the respective analysis variables offer the advantage (individually or in combination) that, on the one hand, typical data that are usually determined during the production of the filler composition and/or composite masses and/or cast workpieces can be used, and no additional measurement data need to be generated or determined for recording the composition dataset. At the same time, this enables the greatest possible reduction in the size of the composition datasets.

It has been found that a database should comprise a minimum number of composition datasets in order to enable a reliable determination of a property of the composite mass and/or the cast workpiece. Therefore, a database should comprise ≥ 50 composition datasets, preferably ≥ 100, more preferably ≥ 150, even more preferably ≥ 175, and most preferably ≥ 200 composition datasets. Determination of a property of the composite mass and/or the cast workpiece can be carried out with a database of this size with an acceptable error tolerance. However, larger databases are conceivable and, in many cases, preferred, for example comprising ≥ 500, preferably ≥ 1000, more preferably ≥ 1500, even more preferably ≥ 2000, and most preferably ≥ 5000 composition datasets. Although the amount of data to be handled increases with such databases, this has proven advantageous in some cases, as the property or properties of the composite mass and/or the cast workpiece can be determined with greater accuracy and robustness on the basis of such a database.

Preferably, more than two composition datasets are used, for example ≥ 5 composition datasets, preferably ≥ 10 composition datasets, more preferably ≥ 20 composition datasets, and most preferably ≥ 50 composition datasets, in order to determine at least one item of information from the above-mentioned group during the computer-implemented determination step. It has been found that with such a number of composition datasets, a meaningful and generally accurate determination becomes possible. For example, the (physico-chemical) property of the first filler composition and/or the first composite mass and/or the first cast workpiece, or the analysis variable characteristic thereof, can be determined with sufficient robustness.

Preferably, during the execution of the method, at least one first analysis variable that characterizes the first property for the first filler composition and/or the first composite mass and/or the first cast workpiece is specified, and during the computer-implemented determination of at least one value of the first composition dataset:

a) the presence or absence of a first component and/or at least one further component in the first composite mass and/or the first cast workpiece, and

b) a proportion value of the first and/or further component in the first composite mass and/or the first cast workpiece is determined.

This process variant enables a user to specify target values for one or more selected analysis variables and, during the process, determine possible composition datasets that are highly likely to result in the production of filler compositions and/or composite masses and/or cast workpieces exhibiting the desired properties correlated with the analysis variables. For example, for kitchen sinks, it would be possible to specify high target values for properties that are particularly relevant for this application, such as temperature change resistance, heat resistance, water resistance, acid resistance, alkali resistance, ductility, and impact toughness. The composition datasets obtained during the implementation of this process variant would enable the production of kitchen sinks that are particularly suitable for use under the specific conditions in this field due to their material properties.

As can be seen from the above example, it is preferable for a composition dataset to comprise several analysis variables in order to give a user, for example in the application described above, the possibility of analyzing several analysis variables or correlated properties of the filler composition and/or the composite and/or cast workpieces.

Preferably, a process variant is provided in which, in addition to or as an alternative to one or more of the variants described above, at least one property, and preferably a plurality of properties, of the first filler composition and/or the first composite mass and/or the first cast workpiece is specified as a target value. Based on this target value, a first component to be added to the first filler composition and/or the first composite mass and/or the first cast workpiece and its proportion value is determined, and/or at least one further component to be added thereto and its respective proportion value is determined. For example, if a specific property, such as high chemical resistance, is required as a target value, this process variant can be used to determine which first component and/or which further component(s) should be added to a filler composition and/or a composite mass and/or a cast workpiece, and in what respective proportions, in order to ensure that the filler composition and/or the composite mass and/or the cast workpiece actually exhibits the target value, namely high chemical resistance in this example.

Preferably, the first composition dataset is determined in such a way that, for at least one (physico-chemical) property, hereinafter also referred to as the target property, and preferably for each specified property, a first component and its proportion value, as well as at least one further component different from the first component and its proportion value, are determined. The determination of the first component and its proportion value, as well as the at least one further component and its proportion value, is preferably carried out as described above, based on another composition dataset and at least one additional composition dataset.

Preference is given to calculating the target property or a correlated analysis variable for the (physico-chemical) property of a first composite mass and/or a first cast workpiece based on the influence of the proportion of the first component and the influence of the proportion of at least one further component. Preferably, one, preferably several, and most preferably each component of the filler composition and/or the composite mass and/or the cast workpiece is assigned a weighting value which correlates with the influence of the respective proportion of the respective component on a change in the target property. For example, a solvent as a component of a composite mass may be assigned a high weighting value for the target property of viscosity, but a low weighting value for the target property of color of the composite mass and/or a cast workpiece producible therefrom.

Preferably, for each specified property (or the analysis variable characterizing that property) of a filler composition and/or a composite mass and/or a cast workpiece, the proportion value of the first component and of at least one further component is determined or calculated separately.

Preferably, at least the second composition dataset and at least one of the further composition datasets include a value (an analysis variable) that is characteristic of the (physico-chemical) target property of the second or further filler composition and/or composite mass and/or cast workpiece characterized by the respective composition dataset (but not of the first composite mass and/or the first cast workpiece). Preferably, a plurality of composition datasets include at least one value (one analysis variable) corresponding to this target property. This enables the assignment of a weighting value to each component of the second or further filler composition and/or composite mass and/or cast workpiece, reflecting the influence of the respective component on the target property.

The use of such composition datasets and the determination of weighting values for the respective components provides the advantage that the amount of data to be transmitted is substantially reduced compared to the prior art or full analyses, and, as explained below, only a significantly smaller number of datasets is required to obtain a robust result. The calculation or determination of the physico-chemical target property can be performed rapidly and does not require extensive test series involving numerous different formulations to supply filler compositions and/or composite masses and/or cast workpieces for subsequent physico-chemical analysis.

In a preferred embodiment, the computer-implemented determination of at least one physic-chemical property characteristic of the first filler composition and/or the first composite mass and/or the first cast workpiece is performed in an entirely computer‑implemented manner. For this purpose, as described above, the composition dataset of the second filler composition and/or a second composite mass and/or the second cast workpiece and at least one further composition dataset of at least one further filler composition and/or at least one further composite mass and/or the at least one further cast workpiece is preferably used as a basis.

Preferably, the determination of at least one property value is performed in a computer-implemented process step. Preferably, this determination is carried out by means of a composite mass formulation determination device, in particular a processor-based device, as described in detail below.

Preferably, the first and/or the second and/or any further composition dataset (in particular, preferably each composition dataset) includes at least one analysis variable and preferably a plurality of analysis variables, for example ≥ 5, preferably ≥ 8, more preferably ≥ 10, and most preferably ≥ 15 analysis variables. Each analysis variable is preferably characteristic of a property of the filler composition and/or composite mass and/or cast workpiece represented by the respective composition dataset. In other words, an analysis variable may serve as an identifier (hereinafter also referred to as “identifier”) of a property of a filler composition and/or composite mass and/or cast workpiece that is of particularly relevant.

Preferably, the property of a filler composition and/or a composite mass and/or a cast workpiece, which is preferably characterized by the analysis variable as described above, is selected from a group comprising: color, color value, color distribution, yellowness, whiteness, gloss, roughness, sound absorption, density, density distribution, bulk density, hardness, particle size distribution, abrasion resistance, wet abrasion resistance, breathability, modulus of elasticity, ductility, (impact) toughness, breaking strength, tear resistance, elasticity, bending stiffness, temperature resistance, thermal shock resistance, melting point, gel time, reactivity, softening temperature, decomposition temperature, combustibility, fire resistance, viscosity, UV resistance, wear resistance, corrosion resistance (e.g., to water, humid air, oxygen), acid resistance, alkali resistance, pH value, (eco)toxicity, pollutant value, health hazard, disposal options, solids content, solvent content, water solubility, shelf life, and diffusion properties. These properties may be used to distinguish filler compositions and/or composite masses and/or cast workpieces from one another and to evaluate their suitability for specific applications.

Preferably, each composition dataset includes, for (at least approximately) every component (or any deliberately added component) contained in the filler composition and/or composite mass and/or cast workpiece characterized by that dataset, a proportion value that is representative of the proportion of this component in the respective filler composition and/or composite mass and/or cast workpiece.

For example, a composition dataset that characterizes a filler composition and/or a composite mass for producing a cast workpiece with a particularly hard surface may include at least one proportion value indicative of a filler with high hardness according to the Mohs scale. Additionally, this composition dataset may comprise one or more further proportion values corresponding to, for example, a solvent, a binder, a filler, a polymer, and/or a preservative. An analysis variable in such an exemplary composition dataset may include, for instance, scratch resistance, hardness, density, viscosity, and/or storage stability.

Preferably, the first component and the second component, and preferably also at least one further component, are each an ingredient of the filler composition and/or the composite mass and/or the cast workpiece, preferably an additive. A solvent may likewise be regarded as a first component, second component, or further component of a composite mass and/or a cast workpiece. In composite masses, the solvent and its proportion are of particular importance, especially with respect to viscosity. The (residual) solvent content may also be relevant for cast workpieces, particularly during shaping and/or curing, as the solvent proportion can significantly influence the time required for demolding.

Preferably, at least one of the first component or the at least one further component and another of the first component or the at least one further component of the filler composition and/or the composite mass and/or the cast workpiece are independently selected from a group comprising: a rheology additive, a pigment, a dye, a color paste, an effect pigment, an emulsifier, a biocide, a hardener, an accelerator, an inhibitor, a filler, a solvent, a polymer, a monomer, a polymerization initiator, an organosilane, a crosslinking agent, a defoamer, a wetting additive, quartz and/or a quartz derivative, a Ca- and/or Mg-containing component, an Al-containing component, a preservative, in particular a film and/or pot preservative, and an inorganic and/or organic filler, which may be mineral, amorphous, or biogenic. Fillers are preferably selected from the group consisting of quartz, cristobalite, feldspar, calcite, dolomite, and wollastonite, which may be present alone or in any ratio in a mixture. In this embodiment, at least two components are independently selected from the above-mentioned group.

In particular, it is preferred that, in addition to the above-mentioned components, at least one, preferably at least two, more preferably at least four, even more preferably at least six, still more preferably at least eight, and particularly preferably at least ten additional components of the first component or the at least one further component of the filler composition and/or the composite mass and/or the cast workpiece are independently selected from a group comprising: a rheology additive, a pigment, a dye, a color paste, an effect pigment, an emulsifier, a biocide, a hardener, an accelerator, an inhibitor, a filler, a solvent, a polymer, a monomer, a polymerization initiator, an organosilane, a crosslinking agent, a defoamer, a wetting additive, quartz and/or a quartz derivative, a Ca- and/or Mg-containing component, an Al-containing component, a preservative, in particular a film and/or pot preservative, and an inorganic and/or organic filler, which may be mineral, amorphous, or biogenic. Fillers are preferably selected from the group consisting of quartz, cristobalite, feldspar, calcite, dolomite, and wollastonite, which may be present alone or in any ratio in a mixture.

In a preferred embodiment, at least one filler composition and/or one composite mass and/or one cast workpiece, and preferably each filler composition and/or composite mass and/or cast workpiece, may be associated with an identification value (referred to as “filler composition ID”, “composite mass ID”, or “cast workpiece ID”), which uniquely characterizes the respective filler composition, composite mass, or cast workpiece. The identification value (generally referred to as “ID”) may also indicate:
a batch of the filler composition, composite mass, or cast workpiece;
a producer of the filler composition, composite mass, or cast workpiece;
a storage condition of the filler composition, composite mass, or cast workpiece;
a transport condition of the filler composition, composite mass, or cast workpiece (such as transport means, transport container, pre-filling of the container, or similar).

This enables any deviation of a property from a target property, caused by these factors, to be traced back using the ID and considered during the computer-implemented determination of at least one value of the first composition dataset.

Preferably, the composition dataset of at least one filler composition and/or one composite mass and/or one cast workpiece is retrieved based on the identification value assigned to the respective composite mass and/or cast workpiece (in particular the “composite mass ID” or “cast workpiece ID”), for example from a database (which may be provided via an external storage device and/or an external server), in which a plurality of pairs of such IDs and the composition dataset assigned to each ID, or data characteristic thereof, are stored.

Preferably, the identification value (ID), in particular the “composite mass ID” or “cast workpiece ID”, is unique, and most preferably, unequivocal for the respective filler composition, composite mass, or cast workpiece. For example, it is conceivable that a first composition dataset of a filler composition and/or a composite mass or a cast workpiece, which has been stored under certain conditions for a predetermined time, is assigned a first identification value, whereas an originally identical filler composition, composite mass, or cast workpiece, stored under different conditions and/or for a different period of time, is assigned a different identification value.

It should be noted that the composition datasets of two originally identical filler compositions, composite masses, or cast workpieces may differ, as one or more analysis variables may have changed due to different storage and/or transport conditions.

Similarly, the composition datasets of a filler composition, a composite mass produced from it, and a cast workpiece manufactured therefrom may differ; however, in some embodiments, it is advantageous to establish a reference between these composition datasets. Such a reference preferably also includes information about the process used to convert the composite mass into the resulting cast workpiece.

The proposed method, particularly the use of a reduced dataset, by utilizing the respective composition datasets for filler compositions, composite masses, and/or cast workpieces, offers the advantage of eliminating or significantly reducing uncertainties or data gaps.

These composition datasets are preferably provided as standard during the production of the respective filler composition, composite mass, or cast workpiece. The amount of data to be transmitted is substantially smaller compared to individual component data. Furthermore, the preferred use of functionally aggregated groups ensures a more robust and reliable result.

In a further preferred embodiment, the determination of at least one property or a correlated analysis variable is based on composition datasets recorded for a plurality of different training filler compositions, composite masses, and/or cast workpieces. These datasets preferably include information on the components contained therein and their respective proportion values within the corresponding filler composition, composite mass, or cast workpiece. Accordingly, a composition dataset is preferably suitable for characterizing a filler composition, composite mass, or cast workpiece with respect to at least one property and at least two components together with their respective proportion values.

The term “training filler composition,” “training composite mass,” or “training cast workpiece” refers to a composition, mass, or workpiece that differs from the filler composition, composite mass, or cast workpiece for which at least one physico-chemical property is to be determined. Preferably, multiple such training compositions, masses, or workpieces are evaluated prior to determining the target property of the first filler composition, composite mass, or cast workpiece, by analysis and/or by consulting formulation data, so that conclusions can be drawn regarding the property of interest. These training compositions, masses, or workpieces are known entities for which measurement data or derived property variables are available that are characteristic of at least one analysis variable.

Compared to the prior art described above, the proposed method offers the advantage of significantly reducing data provision costs. Another advantage is the ability to rely on an established and widely applied standard as the data basis. This method enables robust results and allows the determination of parameters, particularly for the target property, that would otherwise not be computationally predictable or would be more error-prone and subject to greater deviations if determined based on individual components.

Based on the large number of composition datasets recorded for training filler compositions, composite masses, and/or cast workpieces, together with their associated analysis variables that are characteristic of at least one property of these training entities, a machine‑learning model, preferably an artificial neural network, and/or a composite mass formulation model is trained to determine at least one property of a filler composition, composite mass, or cast workpiece. This composite mass formulation model is described in more detail below. In a preferred embodiment, the model is subsequently used to determine at least one physico-chemical property of a first filler composition, a first composite mass, or a first cast workpiece by calculating at least one corresponding property variable.

Preferably, the machine learning composite mass formulation model is based on an artificial neural network, implemented as a deep neural network (DNN) with multiple processing layers, a convolutional neural network (CNN), and/or a recurrent neural network (RNN). Most preferably, the model employs an ensemble learning approach using a random forest (RF), which utilizes decision tree structures that define rules and can be trained independently.

Preferably, the present invention relates to an artificial intelligence / machine-learning (AI/ML) based method for determining at least one property of a filler composition, composite mass, or cast workpiece. In a further preferred embodiment, the determination of at least one physico-chemical property of a first filler composition, composite mass, or cast workpiece, e.g., by means of a composite mass formulation determination device, is performed using a machine- learning formulation model. This model is preferably trainable and comprises a set of parameters adjusted to values learned during a training process based on a set of training data. In another preferred embodiment, the machine learning method is based on a random forest (RF), which uses decision tree structures that define rules and can be trained independently.

Preferably, the method further includes generating a first process dataset containing data for converting a first filler composition into a first composite mass and subsequently into a first cast workpiece. The formulation of the first filler composition or the resulting composite mass may have been determined using the method described above. Preferably, the composite mass is the first composite mass characterized by the first composition dataset derived from the filler composition.

Different methods for converting a filler composition into a composite mass and subsequently into a cast workpiece may result in varying properties of the cast workpiece. Accordingly, multiple process datasets can be associated with a single filler composition and it is corresponding first composite mass. The conversion of the first filler composition into a composite mass according to a process defined by a given process dataset may produce a cast workpiece with properties that differ from those obtained using another process.

The method for generating the first process dataset comprises the following steps:

Recording a second process dataset comprising data relating to a process for converting a second filler composition into a second composite mass and into a second cast workpiece, the second process dataset including at least one value characteristic of a process parameter that influences the first property of a composite mass and/or a cast workpiece as defined by the first analysis variable;

Recording at least one further process dataset, each comprising data relating to a process for converting a further filler composition into a further composite mass and into a further cast workpiece, the further process dataset including at least one value characteristic of a process parameter that influences the first property of a filler composition and/or a composite mass and/or a cast workpiece as defined by the first analysis variable;

- Computer-implemented determination of at least one process data value of the first process dataset based on the second process dataset and at least one further process dataset, the determined process data value being characteristic of a process parameter whose adjustment from another value to the value represented by the process data value enables at least an approximation of the first analysis variable to a target analysis variable, which characterizes a target property of a cast workpiece.

This process variant preferably enables, based on several process datasets and supported by computer-implemented determination of at least one process data value, the identification of a process optimization such that a cast workpiece produced using the modified process exhibits a property whose measurement yields an analysis variable that is closer to a target analysis variable than when measuring this property of a cast workpiece produced using a different process. Thus, the conversion of a first filler composition into a first composite mass and into a first cast workpiece can be optimized at least with respect to the first property characterized by the first analysis variable. This can be achieved without, or at least with significantly reduced and/or less time-consuming experiments and laboratory tests, thereby saving time and resources.

A preferred variant of the method is one in which, during the computer-implemented determination of at least one process data value of the first process dataset, the determined process data value is characteristic of a process parameter selected from a group comprising a time, a temperature, a pressure, a tool, a pretreatment of a tool, a type of application of a reagent, and a sequence of addition of reagents. These process parameters have proven to be particularly suitable for exerting a positive effect on a wide range of properties of a cast workpiece. Therefore, by adjusting a process data value characterizing such a process parameter, it is particularly well possible to change an analysis variable towards a target analysis variable.

Preferably, the second process dataset and/or at least one of the further process datasets each comprise at least three process data values that are characteristic of a process parameter for converting a second or further composite mass into a second or further cast workpiece. Preferably, the second process dataset and/or at least one of the further process datasets, independently of any other process dataset, comprise at least five, more preferably at least seven, and most preferably at least ten process data values. This enables several process parameters, which may influence each other, to be considered and/or weighted together. As a result, multiple process data values can be jointly optimized, preferably taking mutual interactions into account as described above, so that an even more improved adjustment of the process parameters characterized thereby is achieved, and thus also an improved approximation of the analysis variable towards a target analysis variable.

The process data values described above are preferably determined during the conversion of a second or further composite mass into a second or further cast workpiece. Frequently, some process parameters are already determined during the conversion of a composite mass into a cast workpiece. These values are often stored for documentation purposes and/or quality assurance. By determining additional process data values and/or accessing existing databases, it is possible to provide process datasets for use in this process variant. In addition or as a supplement to this, it would also be possible to subsequently supplement individual or multiple process datasets and/or individual process data values of one or more process datasets, for example by extrapolation, estimation, and/or the use of a suitable artificial intelligence system.

For the step of computer-implemented determination of at least one process data value of the first process dataset, a process data value determination model is preferably used. Preferably, such a machine-learning-based process data value determination model is implemented using an (artificial) neural network. The neural network is preferably designed as a deep neural network (DNN), in which the parameterizable processing chain comprises a plurality of processing layers, and/or as a convolutional neural network (CNN), and/or as a recurrent neural network (RNN). Particularly preferably, the process data value determination model is based on a random forest model (RF). Random forest models operate with decision tree structures that establish specific rules and can be trained separately. These models work particularly well with small datasets, and the decision-making process is easily interpretable.

The machine-learning-based process data value determination model is preferably trained using predefined training data, whereby the training parameterizes the parameterizable processing chain. Preferably, the training data include the above-mentioned process data values, which were determined during previous conversions of (preferably filler compositions into) composite masses and subsequently into cast workpieces.

It is also possible that the process data value determination model forms part of the composite mass formulation model described in more detail below and thus also comprises information about possible processes for converting (preferably filler compositions into) composite masses and subsequently into cast workpieces.

Furthermore, in some embodiments it is preferred that the process data value determination model and the composite mass formulation model are similar in one or more aspects, for example, that they are structured, trained, stored, and provided in an analogous manner. Accordingly, all embodiments and methods described below for the composite mass formulation model shall also be deemed disclosed for the process data value determination model.

The same applies to a process data value determination device not described in detail here, which is preferably analogous in structure and configuration to the composite mass formulation determination device described below. However, the process data value determination device does not have to implement all aspects described below for the composite mass formulation determination device together and/or in an analogous manner. Rather, it is possible that the process data value determination device may be implemented independently of the composite mass formulation determination device, with only some of the aspects described below for the composite mass formulation determination device being realized, while others are not.

The machine-learning-based composite mass formulation model is preferably trained using predefined training data, whereby the training parameterizes the processing chain.

Preferably, the training data comprise at least one second composition dataset relating to a second filler composition and/or a second composite mass and/or a second cast workpiece, wherein the second composition dataset includes at least one analysis variable and one proportion value of a second component and preferably a plurality of proportion values of further components, as well as at least one further composition dataset relating to at least one further filler composition and/or a further composite mass and/or a further cast workpiece, wherein the at least one further composition dataset includes at least one further analysis variable and one further proportion value of a further component and preferably a plurality of further proportion values of further components. Preferably, the second and/or further analysis variable is characteristic of at least one property of the filler composition and/or composite mass and/or cast workpiece characterized by the respective composition dataset.

Preferably, the composite mass formulation model or the (artificial) neural network is supplied with the data to be processed, in particular the first analysis dataset mentioned above and at least one further composition dataset (or data derived therefrom) as input variables. Preferably, the composite mass formulation model, the artificial neural network, or the random forest model maps the input variables to output variables depending on a parameterizable processing chain.

Preferably, at least one output variable is selected that is characteristic of a property of the filler composition and/or the composite mass and/or the cast workpiece (for example, a density value or a viscosity value).

However, it is also conceivable to specify a target value for a physico-chemical property of the filler composition and/or the composite mass and/or the cast workpiece and to use the method to determine descriptors for a filler composition and/or a composite mass and/or a cast workpiece which, according to this determination, are likely to exhibit the specified target value of the property. In this variant of the method, a target value of the physico-chemical property is specified, and the components of the filler composition and/or the composite mass and/or the cast workpiece, as well as the proportion values associated with these components, are determined. This process variant is particularly advantageous when searching for formulations for a filler composition and/or a composite mass and/or a cast workpiece that should exhibit a specific target property.

In a preferred method, the training process of the composite mass formulation model uses training data that includes at least one analysis variable and values characteristic of components of a filler composition and/or a composite mass and/or a cast workpiece, together with proportion values assigned to these components. Such data may be obtained through analysis (preferably for the analysis variables) and/or during the production of the composite masses and/or cast workpieces on which the training data is based (e.g., from formulation specifications or monitoring systems of the equipment used, preferably for the proportion values and associated components of the filler composition and/or the composite mass and/or the cast workpiece). It is also conceivable to use training data derived from filler compositions and/or composite masses and/or cast workpieces from different batches (optionally from different suppliers and/or components from various sources). This approach offers the advantage of generating a large amount of training data within a short time.

Preferred training data is supplemented with additional attributes, such as an analysis attribute (which may include one or more parameters characteristic of the type, method, or service provider used to determine this analysis variable), a manufacturer attribute, a storage attribute (which may include one or more parameters characteristic of the type, duration, temperature, storage container, or other storage-related property), and/or a transport attribute (which may include one or more parameters characteristic of the type, duration, route, temperature, transport container, or other transport-related property).

Preferably, the analyzed datasets, optionally together with their associated attributes (e.g., analysis, manufacturer, storage, and/or transport attributes), are stored and/or used as training datasets, particularly on a non-volatile storage device. This approach enables the provision of a large number of training datasets.

By varying the proportion values of one or more components and correlating them with one or more analysis variables, it is theoretically possible to establish correlations between the proportion values of individual components and the analysis variables, or between these proportion values and a property of a filler composition, a composite mass, or a cast workpiece.

A neural network trained in this way (serving as the composite mass formulation model) is preferably used, with a Random Forest model being particularly preferred. Training is typically performed using supervised learning; however, unsupervised learning, reinforcement learning, or stochastic learning may also be applied.

The composite mass formulation model is preferably provided with the processable data, specifically the first and at least one additional composition dataset, or data characteristic of or derived from these, as input variables. The model then maps these input variables to output variables via a configurable processing chain.

Preferably, at least one output variable is selected that represents a property of a filler composition and/or a composite mass and/or a cast workpiece, or a value characteristic of a filler composition and/or a composite mass and/or a cast workpiece.

The output variable preferably also includes data characteristic of a first component and at least one additional component different from the first component, and more preferably also their respective proportion values in a filler composition and/or a composite mass and/or a cast workpiece, or values characteristic of these proportions.

Preferably, the composite mass formulation model based on machine learning is or was trained using predefined training data, whereby the training process parameterizes the configurable processing chain. In a preferred method, the training process of the composite mass formulation model uses training data that includes historically recorded data, in particular for formulations of filler compositions and/or composite masses and/or cast workpieces, and at least one (physico-chemical) property of a filler composition and/or a composite mass and/or a cast workpiece. This offers the advantage that a large amount of training data can be generated in a short time.

The training data preferably contains, as a classification feature, an indication of whether an analysis variable meets a specified target value (or falls within a defined target range) or does not meet the target value (or falls outside the defined range).

In a further preferred method, the determined property value is transmitted to an output device, for example a screen and/or a (particularly mobile) user terminal. Additionally or alternatively, the determined property value may be stored on an external and/or cloud-based storage device (as described above), preferably together with a generated identifier assigned to the filler composition and/or the composite mass and/or the cast workpiece (such as a filler composition ID, composite mass ID, or cast workpiece ID), and preferably provided for later output to a user via an output device. referably, each manufactured product, preferably each filler composition and/or each composite mass and/or each cast workpiece, is assigned a unique identification value (filler composition ID, composite mass ID, or cast workpiece ID), as described above. The analysis datasets determined or generated during the production of the filler composition and/or the composite masses and/or the cast workpieces (typically as standard) are preferably stored together with the identification value in the database described above, preferably on an external server (in relation to the composite mass formulation determination device).

Preferably, each manufactured product, preferably each filler composition and/or each composite mass and/or each cast workpiece, is assigned a unique identification value (filler composition ID, composite mass ID, or cast workpiece ID), as described above. The analysis datasets determined or generated during the production of the filler composition and/or the composite masses and/or the cast workpieces (typically as standard) are preferably stored together with the identification value in the database described above, preferably on an external server (in relation to the composite mass formulation determination device).

The invention further relates to a computer-implemented method for generating a trained composite mass formulation model based on machine learning for determining at least one first property of a first filler composition and/or a first composite mass and/or a first cast workpiece producible therefrom, based on the use of composition datasets, each of which is characteristic of a filler composition and/or a composite mass and/or a cast workpiece. The composition dataset for the respective composite mass and/or cast workpiece comprises at least one analysis variable and a proportion value of a component, wherein the analysis variable is characteristic of at least one property of the filler composition and/or composite mass and/or cast workpiece characterized by the respective composition dataset, comprising:

Providing a trainable composite mass formulation model for machine learning, which comprises a set of trainable parameters and which, based on at least one composition dataset recorded for a filler composition and/or a composite mass and/or a cast workpiece or data derived therefrom as input variables, generates at least one analysis variable characteristic of a property of the first filler composition and/or the first composite mass and/or the first cast workpiece as an output variable;

Generating a training dataset comprising a plurality of recorded and/or determined composition datasets for training filler compositions and/or training composite masses and/or training cast workpieces, which are used to create the training dataset, as well as measurement data or analysis variables derived therefrom that are characteristic of at least one property of the respective training filler compositions and/or training composite masses and/or training cast workpieces;

Training the composite mass formulation model based on the training dataset.

The composite mass formulation model generated in this manner is preferably employed to determine at least one analysis variable indicative of a property of a first filler composition, composite mass, or cast workpiece. This determination may preferably be performed as part of the method described above and/or using the composite mass formulation determination device described below.

The present invention also relates to a composite mass formulation determination device, preferably implemented as a processor-based system, for determining at least one property, preferably a physico-chemical property, of a first filler composition, a first composite mass, or a first cast workpiece.

According to the invention, the composite mass formulation determination device is configured and designed to record a second composition dataset relating to a second filler composition and/or a second composite mass and/or a second cast workpiece, as well as at least one further composition dataset relating to at least one additional filler composition and/or an additional composite mass and/or an additional cast workpiece. Each composition dataset comprises at least one second or further analysis variable and at least one second or further proportion value of a second or further component, and preferably a plurality of proportion values of additional components. Each analysis variable is characteristic of at least one property of a second or further filler composition and/or a second or further composite mass and/or a second or further cast workpiece (GWS2–n), and each proportion value is characteristic of the quantity fraction of the second and/or further components contained in the respective filler composition and/or composite mass and/or cast workpiece. The composite mass formulation determination device is further configured and designed to determine, by means of a processor unit, at least one value of a composition dataset that contains data for formulating a first filler composition or a first composite mass for producing a first cast workpiece, based on the second composition dataset for the second filler composition and/or the second composite mass and/or the second cast workpiece and at least one further composition dataset for the at least one additional composite mass and/or additional cast workpiece.

Each composition dataset is preferably structured in accordance with the previously described preferred embodiment.

It is therefore also proposed within the scope of the inventive composite mass formulation determination device that, by using the composition datasets, data records that are already largely available (and optionally supplemented by analysis data) are utilized, in particular those that are typically provided during the manufacture of filler compositions and/or composite masses and/or cast workpieces or determined during the ongoing process. The amount of data to be transmitted is significantly smaller compared to individual component data. Furthermore, the use of functionally aggregated groups, as described above in the context of the method, advantageously produces a more robust result.

The composite mass formulation determination device is preferably configured, suitable, and/or designed to execute the above-described method for generating a first composition dataset, which comprises data for formulating a first composite mass for producing a first cast workpiece, as well as one or all of the process steps already described above in connection with the method (or a preferred embodiment of the method), individually or in combination. Conversely, the method can be carried out with all the features described for the composite mass formulation determination device, individually or in combination.

The composite mass formulation determination device is further preferably configured and designed to use a trained composite mass formulation model generated according to the method described above (preferably with respect to a first composition dataset characterizing a first filler composition and/or the first composite mass and/or a first cast workpiece). For this purpose, the composite mass formulation determination device is preferably supplied with a second composition dataset and at least one additional composition dataset. The device is further configured and designed to determine, from the analysis datasets provided to it, a composition dataset that includes at least one first analysis variable characterizing the (preferably physico-chemical) property of the first filler composition and/or the first composite mass and/or the first cast workpiece.

The term external server as described above refers in particular to a server external to the composite mass formulation determination device, specifically a backend server. The external server may, for example, be the backend of a manufacturer of filler compositions and/or composite masses and/or cast workpieces, or of a distributor of composite masses and/or cast workpieces, a raw material supplier for products in the cast workpiece industry, or a service provider configured to determine and/or generate and/or manage and/or store composition datasets relating to filler compositions and/or composite masses and/or cast workpieces. The functions of the backend or external server may be performed on external server farms. The external server may be implemented as a distributed system. The external server and/or the backend may be cloud-based.

The present invention also relates to a plant for producing a composite mass and/or a cast workpiece, comprising a composite mass formulation determination device as described above in accordance with one embodiment (described above).

Preferably, the plant is equipped with a corresponding interface which enables the transmission of at least one composition dataset and/or the selection of a desired analysis variable which characterizes the (target) property of the filler composition and/or the composite mass and/or the cast workpiece.

The present invention further relates to a plant for producing a composite mass and/or a cast workpiece, comprising a composite mass formulation determination device as described above in accordance with a previously described embodiment.

The plant is preferably equipped with a corresponding interface that enables the transmission of at least one composition dataset and/or the selection of a desired analysis variable that characterizes the (target) property of the filler composition and/or the composite mass and/or the cast workpiece.

The present invention further relates to a computer program or computer program product comprising program means, in particular program code, which performs at least some and preferably all of the method steps of the inventive methods (method for generating a first composition dataset comprising data for formulating a first filler composition and/or a first composite mass for producing a first cast workpiece; method for generating a trained machine-learning-based composite mass formulation model; use of the trained machine-learning-based composite mass formulation model to determine at least one analysis variable characteristic of at least one property of a first filler composition and/or a first composite mass and/or a first cast workpiece) and preferably represents or encodes one of the described preferred embodiments and is designed to be executed by a processor device.

The present invention further relates to a data storage device on which at least one embodiment of the inventive computer program or a preferred embodiment of the computer program is stored.

Further advantages and embodiments are apparent from the accompanying drawings:

### BRIEF DESCRIPTION OF THE DRAWINGS

FIG. 1 a schematic representation of a sequence of a method according to the invention in accordance with a preferred embodiment, and

FIG. 2 a schematic representation of a multi-stage sequence of a method according to the invention in accordance with a preferred embodiment.

### DETAILED DESCRIPTION OF THE INVENTION

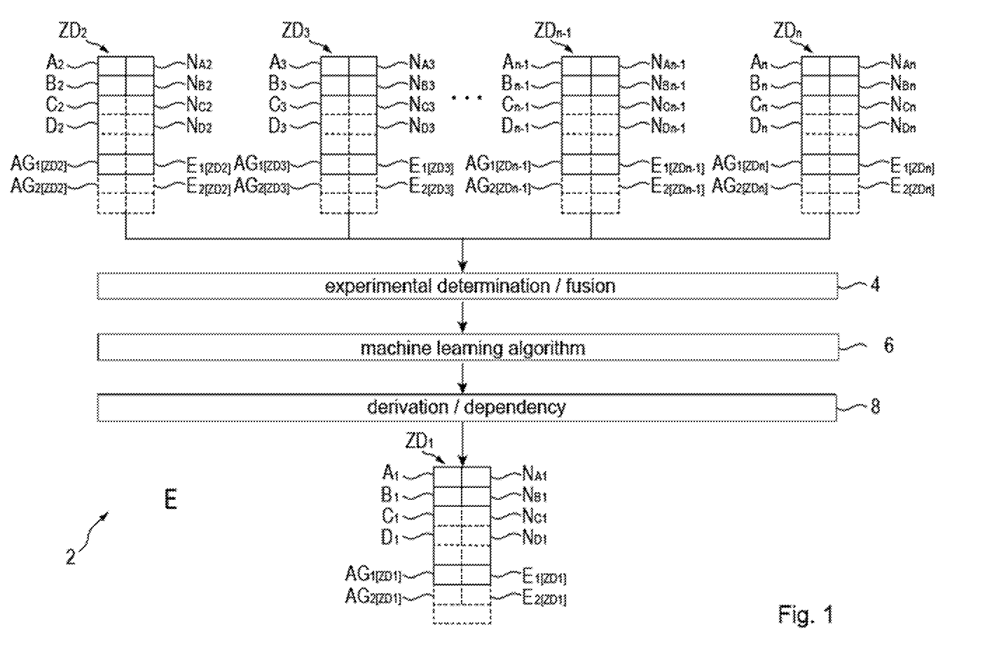

FIG. 1 schematically illustrates a sequence of a method (2) according to a preferred embodiment of the invention. The reference signs ZD₁ – ZDₙ each denote a composition dataset, without specifying whether the respective composition dataset characterizes a filler composition, a composite mass, or a cast workpiece. Each composition dataset ZD₁– ZDₙ comprises descriptors for a respective filler composition, composite mass, or cast workpiece. These descriptors may be obtained by analysis or may be known from the formulation of the respective filler composition, composite mass, or cast workpiece. The number, type, and value of the descriptors may differ or be identical between two composition datasets.

As shown in FIG. 1, the objective of the method is to determine the descriptors for a first composition dataset ZD₁, which comprises data for the formulation of a first filler composition and/or a first composite mass and/or a first cast workpiece.

The first composition dataset ZD₁ comprises information relating to a first component A₁ and at least one further component B₁, which is different from the first component A₁, as well as a proportion value N_{A1} of the first component A₁ and a proportion value N_{B1} of the second component B1. The proportion values N_{A1}, N_{B1}, … N_{An} each represent the proportion of the components A₁, B₁, … X₁ in the first filler composition, the first composite mass, and/or the first cast workpiece.

Likewise, each of the further composition datasets ZD₂ – ZDₙ comprises information relating to a first component A₂ and at least one further component B₂, which is different from the first component A₂, as well as a proportion value N_{A2} of the first component A₂ and a proportion value N_{B2} of the second component B₂. The components A_{2–n} to X_{2–n} stored in a composition dataset ZD₂ – ZDₙ may be identical or different. For example, it is conceivable that a first component in one of the composition datasets ZD₂– ZDₙ defines quartz of a specified particle size distribution as a possible filler for a filler composition, a composite mass, and/or a cast workpiece, whereas quartz of this particle size distribution is not contained in another filler composition, composite mass, and/or cast workpiece. This could be represented by the variable of component Aₓ being assigned differently in another composition dataset ZD₂ – ZDₙ than in composition dataset ZD_{b}, or by the variable of component Aₓ being assigned identically in all composition datasets ZD_{b} – ZDₙ, but the proportion value N_{Ax} for one or some of the composition datasets ZD₁ – ZDₙ characterizing the absence of component Ax in the filler composition, composite mass, and/or cast workpiece described by that composition dataset by a value representing a mass fraction of zero.

The number of composition datasets ZD₂ – ZDₙ is not limited. However, a large number is advantageous for improving the robustness of the method. It has been found that a set of more than 50 composition datasets ZD₂ – ZDₙ (n = 50) may already be sufficient to achieve reasonably good results. Nevertheless, a larger number of analysis datasets, such as ≥ 150 analysis datasets (n ≥ 150), is advantageous, as stated above, with regard to the quality and robustness of a calculation or prediction of a property of a filler composition, composite mass, or cast workpiece.

The composition dataset ZD₁ comprises information on at least one property E₁, E₂, … Eₙ of the first filler composition and/or the first composite mass and/or the first cast workpiece, which can be characterized by a first analysis variable AG₁, AG₂, … AGₙ, wherein FIG. 1 does not specify whether the composition dataset ZD₁ defines a filler composition, a composite mass, or a cast workpiece.

Furthermore, at least one second and/or further composition dataset ZD₂, ZD₃, … ZDₙ relating to a second and/or further filler composition, composite mass, or cast workpiece likewise comprises at least one second and/or further analysis variable AG_{1–n[ZD2–ZDn]},each of which is characteristic of at least one property E_{1–n[FM2–FMn]},E_{1–n[KM2–KMn]},E_{2–n[GWS2–GWSn]} of the filler composition, composite mass, or cast workpiece characterized by the respective composition dataset ZD₂, … ZDₙ. It is not necessary for an analysis variable AG_{1–n[ZD2–ZDn]} that correlates with a specific property to be present in all composition datasets ZD₂ – ZDₙ. It is also conceivable that, for subsequent process steps, only those composition datasets ZD₂– ZDₙ (or with higher weighting) are considered that contain an analysis variable AG_{1–n[ZD2–ZDn]} correlating with a specific (target) property E_{1–n[ZD1–ZDn]}.

Each of the composition datasets ZD₁ – ZDₙ therefore preferably comprises at least one analysis variable AG_{1[ZD2–ZDn]}.Multiple analysis variable AG_{1–n[ZD2–ZDn]} are advantageous, as stated above, wherein FIG. 1 illustrates only one additional analysis variable AG_{2[ZD2–ZDn]} for each analysis dataset, and further analysis variables AG_{3–n[ZD2–ZDn]} are merely indicated by dashed areas.

An analysis variable AG_{1–n[ZD2–ZDn]} represents, in each case, a property E_{1–n[ZD1–n]} of a filler composition, a composite mass, or a cast workpiece. For example, this may be a color location, hardness, viscosity, density, abrasion resistance, or another of the properties mentioned above. This property may already be known and included when creating the composition datasets ZD₁ – ZDₙ, or it may be added to the respective composition dataset ZD₁ – ZDₙ subsequently, for example after a corresponding analysis of the relevant filler composition, composite mass, or cast workpiece with respect to this property. It is not necessary for every composition dataset ZD₁ – ZDₙ to contain a value for each property E_{1–n[ZD1–n]}.It is also conceivable that individual composition datasets ZD₁ – ZDₙ do not contain a value for a specific property E_{1–n[ZD1–n]}.

The indices “FM₁–FMₙ”, “KM₁–KMₙ” and “GWS₁–GWSₙ” refer to a first to n-th filler composition (FM₁–FMₙ), a first to n-th composite mass (KM₁–KMₙ), and a first to n-th cast workpiece (GWS₁ … GWSₙ), respectively. If it is irrelevant whether it concerns a filler composition, a composite mass, or a cast workpiece, or if the above description is intended to be generic, the index “ZD₁–ZDₙ” is used. The index ZD₁–ZDₙ then generally stands for a first to n-th filler composition, a first to n-th composite mass, or a first to n-th cast workpiece.

It is proposed to use the composition datasets ZD₂ – ZDₙ and the descriptors contained therein (A_{2–n}, B_{2–n}, …; N_{A2–n}, N_{B2–n[ZD1–ZDn],} …), including the analysis variables AG_{1[ZD2–ZDn]} – AG_{n[ZD2–ZDn]},for a machine learning algorithm 6. For this purpose, the process step identified by reference sign 4 provides for an experimental determination and/or fusion of the composition datasets ZD₂– ZDₙ and the analysis variables AG_{1[ZD2–ZDn]} – AG_{n[ZD2–ZDn]}.

The machine learning algorithm 6 is then trained using a number of training datasets, and for the respective target value, which correlates with a property E_{1[ZD1]}, E_{2[ZD1]},…; E_{n[ZD1]} of the first filler composition FM₁, the first composite mass KM₁, or the first cast workpiece GWS₁, a derivation function is generated as a function of the determined parameters. In FIG. 1, reference sign 8 denotes the derivation of such a dependency.

The training dataset is used to train the machine learning algorithm 6 or to train a neural network, i.e., for machine learning to predict the physico-chemical property E_{1[ZD1]},E_{2[ZD1]}, …; E_{n[ZD1]} of the first filler composition FM₁, the first composite mass KM₁, or the first cast workpiece GWS₁ as a function of the composition datasets ZD₂, … ZDₙ. From this (by the trained neural network or the trained machine learning algorithm), a prediction function is obtained, which is preferably used to predict at least one physico-chemical property E_{1[ZD1]},E_{2[ZD1]}, …; E_{n[ZD1]} of the first filler composition FM₁, the first composite mass KM₁, or the first cast workpiece GWS₁. Based on the composition dataset ZD₁ correlating with the first filler composition FM₁, the first composite mass KM₁, or the first cast workpiece GWS₁ and the prediction function, an expected value for at least one physico-chemical property E_{1[ZD1]}, E_{2[ZD1]},…; E_{n[ZD1]} of this first filler composition FM₁, the first composite mass KM₁, or the first cast workpiece GWS₁ can be determined.

It is likewise conceivable to specify a target value of the physico-chemical property E_{1[ZD1]} and enable the system to determine descriptors for a filler composition, a composite mass, or a cast workpiece that, according to this prediction, is likely to exhibit the specified property E_{1[ZD1].} In this variant of the method, a target value of the physico-chemical property E_{1[ZD1]} is therefore specified, and the components (A₁, B₁, C₁, …) as well as the proportion values (N_{A1}, N_{B1}, N_{C1}, …) associated with these components are determined. A filler composition with the filler composition components (A_{Z}, B_{Z}, C_{Z}, …) in the proportions according to the proportion values (N_{Az}, N_{Bz}, N_{Cz}, …) will, according to this prediction, exhibit the specified physico-chemical property E_{1[ZD1]} with the target value.

Based on at least some of the above-described second composition dataset ZD₂ and at least one of the further composition datasets ZD₃, … ZDₙ, this variant of the method performs a computer-implemented determination of at least one value of the first composition dataset ZD₁. The at least one value determined in this process is characteristic of:
a) the presence or absence of a first component (A₁) and/or at least one further component B₁ in the first filler composition FM₁ and/or a first composite mass KM₁ and/or the first cast workpiece GWS₁,
b) a proportion value N_{A1}, N_{B1}, … N_{X1} of the first and/or further component A₁, B₁, … X₁ in the first filler composition FM₁ and/or a first composite mass KM₁ and/or the first cast workpiece GWS₁, or
c) at least one first property (E_{1[ZD1]},E_{2[ZD1]},…; E_{n[ZD1]})of the first filler composition FM₁, the first composite mass KM₁, or the first cast workpiece GWS₁, which can be characterized by the first analysis variable AG_{1[ZD1]}, AG_{2[ZD1]},…; AG_{n[ZD1]}.

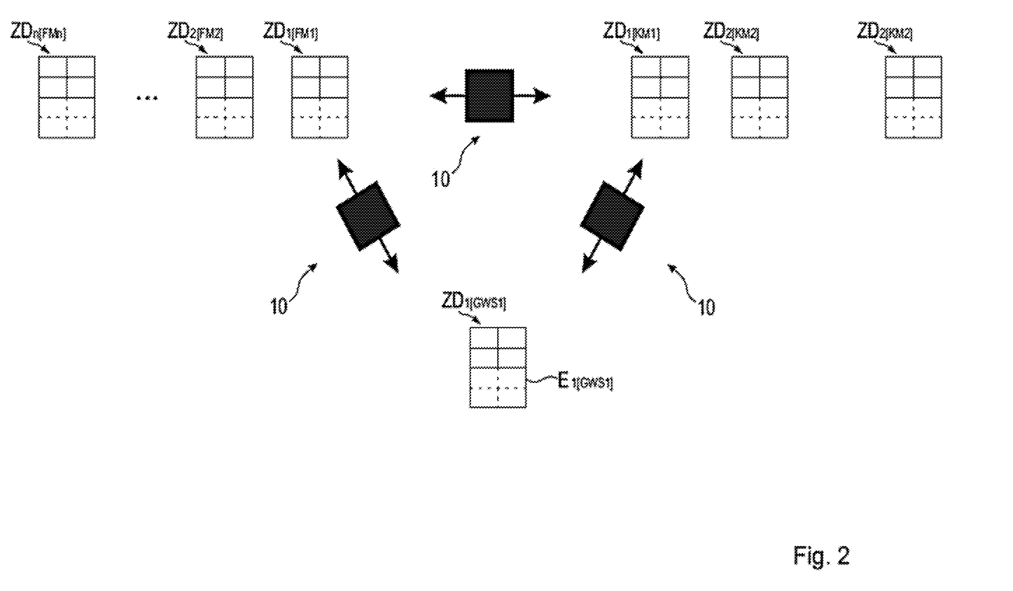

FIG. 2 shows a schematic representation of a multi-stage sequence of a method according to the invention in accordance with a preferred embodiment. In each case, the step of computer-implemented determination of at least one value of a composition dataset ZD_{1[FM1/KM1/GWS1]} is schematically illustrated and identified by reference sign 10. The double arrow indicates that the method can be used on both sides of the arrow for determining components and/or properties. For example, components of a first composite mass KM₁ could be specified, and a property E_{1[GWS1]} of a cast workpiece GWS₁ producible therefrom could be determined.

Alternatively, as outlined above, it would also be conceivable to specify a desired (target) property E₁, for example of a cast workpiece GWS₁, and allow the system to determine those components of a first composite mass KM₁ that can be processed into a cast workpiece GWS₁ having the desired (target) property E₁.

Likewise, it would be conceivable that a desired (target) property E₁, for example of a cast workpiece GWS₁, is specified and a first filler composition FM₁ is determined that, within the framework of a specific formulation and/or a specific process, can be converted into the cast workpiece GWS₁ with the desired (target) property E₁.

The method can also be used to determine process steps that are necessary for converting a composite mass KM₁ into a cast workpiece GWS₁, a filler composition FM₁ into a cast workpiece GWS₁, or a filler composition FM₁ into a composite mass KM₁, so that the product exhibits the desired (target) property E₁. These process data, which can likewise be determined by a computer-implemented method, are schematically represented in FIG. 2 by the black square in the area of the double arrows.

The applicant reserves the right to claim all features disclosed in the application documents as essential to the invention, provided they are new individually or in combination with respect to the prior art. It is further noted that the figure also describes features that may be advantageous on their own. The skilled person will immediately recognize that a specific feature described in the figures may also be advantageous without adopting additional features from that figure. Furthermore, the skilled person will recognize that advantages may also result from a combination of one or more features shown in a figure with one or more features described in the text.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with "including," "containing," or "characterized by," and is inclusive or open-ended and does not exclude additional, unrecited elements or method steps. As used herein, "consisting of" excludes any element, step, or ingredient not specified in the claim element. As used herein, "consisting essentially of" does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

1. A method for generating a first composition dataset (ZD₁) that comprises data for a formulation of a first filler composition (FM₁) and/or a first composite mass (KM₁) for production of a first cast workpiece (GWS₁), wherein the first composition dataset (ZD₁) comprises:
information on a first component (A1) and at least one further second component (B1) different from the first component (A1)
a proportion (NA1) of the first component (A1) and a proportion (NB1) of the second component (B1) in the first filler composition (FM1) and/or a first composite mass (KM1) and/or the first cast workpiece (GWS1), and
information on at least one parameter determined by a first analysis variable (AG1[KM1], AG2[KM1], …; AG1[GWS1], AG2[GWS1], …) characteristic property (E1[KM1], E2[KM1], …; E1[GWS1], E2[GWS1], …) of the first filler composition (FM1) and/or the first composite mass (KM1) and/or the first cast workpiece (GWS1),
wherein the method comprises the steps:
recording a second composition dataset (ZD2) for a second filler composition
(FM2) and/or a second composite mass (KM2) and/or a second cast workpiece (GWS2),
wherein the second composition dataset (ZD2) comprises at least one second analysis variable (AG1[KM2], AG1[GWS2]) and a proportion (NA2) of a second component (A2) and a plurality of proportions (NB2, NC2, ...) of further components (B2, C2, ...),
recording at least one further composition dataset (ZD3, ... ZDn) for at least one further filler composition (FM3, ... FMn) and/or a further composite mass (KM3, ... KMn) and/or a further cast workpiece (GWS3, … GWSn), wherein the at least one further composition dataset (ZD3, … ZDn) comprises at least one further analysis variable (AG1-n[FM3-FMn], AG1-n[KM3-KMn], AG1-n[GWS3-GWSn]) and a further proportion value (NA3, … NAn) of a further component (A3, … An) and, optionally, a plurality of further proportion values (NB3, … NBn, NC3, … NCn, …) of further components (B3-n, C3-n, ...),
wherein the second and/or further analysis variable (AG2-n[FM3-FMn], AG2-n[KM3-KMn], AG2-n[GWS3-GWSn]) is characteristic of at least one property (E2-n[KM3-KMn], E2-n[GWS3-GWSn]) of the filler composition (FM2, … FMn) and/or composite mass (KM2, … KMn) and/or cast workpiece (GWS2, … GWSn) as characterized by the respective composition dataset (ZD2, … ZDn), wherein the second and/or further proportion value (NA2, … NAn, NB2, … NBn, …) is characteristic of the proportion of the first and/or further component (A2-n, B2-n, …) contained in the filler composition (FM2, … FMn) and/or composite mass (KM2, … KMn) and/or cast workpiece (GWS2, … GWSn) as characterized by the respective composition dataset (ZD2, … ZDn); and
computer-implemented determination of at least one value of the first composition dataset (ZD1) based on the second composition dataset (ZD2) and at least one further composition dataset (ZD3, ... ZDn), wherein the determined value is selected from information from a group of information:
a) about the presence or absence of a first component (A
b) about a proportion value (NA1, NB1, ...) of the first and/or further components (A1, B1, ...) in the first filler composition (FM1) and/or a first composite mass (KM1) and/or the first cast workpiece (GWS1), and
c) relating to the at least one first property (E1[KM1], E2[KM1], ...; E1[GWS1], E2[GWS1], ...) of the first filler composition (FM1) and/or the first composite mass (KM1) and/or the first cast workpiece (GWS1) as characterized by the first analysis variable (AG1[KM1], AG2[KM1], ...; AG1[GWS1], AG2[GWS1], ...).1[KM1]2[KM1]1[GWS1] 2[GWS1]1111[KM1] 2[KM1]1[GWS1] 2[GWS1]

2. The method according to claim 1, wherein the at least one first analysis variable (AG_{1[KM1]}AG_{2[KM1]}…; AG_{1[GWS1]} AG_{2[GWS1]} ...), which characterizes the first property (E_{1[KM1]} E_{2[KM1]} ...; E_{1[GWS1]}E_{2[GWS1]} ...) for the first filler composition (FM₁) and/or the first composite mass (KM₁) and/or the first cast workpiece (GWS₁), is specified and, during the computer-implemented determination of at least one value of the first composition dataset (ZD₁)
a) the presence or absence of a first component (A1) and/or at least one further component (B1) in the first filler composition (FM1) and/or in the first composite mass (KM1) and/or the first cast workpiece (GWS1), and
b) a proportion value (NA1, NB1, ...) of the first and/or further component (A1, B1, ...) in the first filler composition (FM1) and/or the first composite mass (KM1) and/or the first cast workpiece (GWS1) is determined.

3. The method according to claim 1, wherein at least one of the first component (A₁) or the at least one further component (B₁ C₁ ...) and another of the first component (A₁) or the at least one further component (B₁ C₁ ...) is independently selected from a group comprising a rheology additive, a pigment, a dye, a color paste, an effect pigment, an emulsifier, a biocide, a hardener, an accelerator, an inhibitor, a filler, a solvent, a polymer, a monomer, a polymerization initiator, an organosilane, a crosslinker, a defoamer, a wetting additive, quartz and/or quartz derivative, a Ca- and/or Mg-containing component, an Al-containing component, a preservative, in particular a film and/or pot preservative, an inorganic and/or organic filler, which may be mineral, amorphous, or biogenic, wherein at least one further component of the first component (A1) or the at least one further component (B₁ C₁ ...) is selected from a group comprising a rheology additive, a pigment, a dye, a color paste, an effect pigment, an emulsifier, a biocide, a hardener, an accelerator, an inhibitor, a filler, a solvent, a polymer, a monomer, a polymerization initiator, an organosilane, a crosslinker, a defoamer, a wetting additive, quartz and/or quartz derivative, a Ca- and/or Mg-containing component, an Al-containing component, a preservative, in particular a film and/or pot preservative, an inorganic and/or organic filler, which may be mineral, amorphous, or biogenic.

4. The method according to claim 1, wherein the first cast workpiece (GWS₁) is selected from a group comprising a kitchen sink, a kitchen worktop, a washbasin, a shower tray, a shower cubicle, a toilet, a bathtub, a wall- tile, a floor- slab, a floor covering, a piece of furniture, and a partition wall.

5. The method according to claim 1, wherein the method further comprises generating a first process dataset (VD₁) comprising data relating to a method for converting a first filler composition (FM₁) and/or a first composite mass (KM₁) into a first cast workpiece (GWS₁), wherein the method for generating the first process dataset (VD₁) comprises the steps:
recording of a second process dataset (VD2), which comprises data relating to a process for converting a second filler composition (FM2) into a composite mass (KM2) and into a second cast workpiece (GWS2), wherein the second process dataset (VD2) contains at least one value, which is characteristic of a process parameter, that has an effect on the first property (E1) of a filler composition (FMX) and/or a composite mass (KMx) and/or a cast workpiece (GWSx) characterized by the first analysis variable (AG1),
recording at least one further process dataset (VD3, ... VDn), each of which contains data relating to a process for converting a further filler composition (FM3, ...FMn) and/or a further composite mass (KM3, ... KMn) into a further cast workpiece (GWS3, ... GWSn), wherein the further process dataset (VD3, … VDn) contains at least one value, which is characteristic of a process parameter, which has an effect on the first property (E1) of a filler composition (FMx) and/or a composite mass (KMx) and/or a cast workpiece (GWSx) characterized by the first analysis variable (AG1),
computer-implemented determination of at least one process data value (VDW1) of the first process dataset (VD1) based on the second process dataset (VD2) and at least one further process dataset (VD3, ... VDn), wherein this process data value (VDW1) is characteristic of a process parameter, whose adjustment from another value to the value characterized by the process data value (VDW1) enables at least an approximation of the first analysis variable (AG1) to a target analysis variable, which is characteristic of a target property of the first cast workpiece (GWS1).

6. The method according to claim 5, wherein the at least one process data value (VDW₁), which is determined during the computer-implemented determination of the at least one process data value (VDW₁) of the first process data record (VD₁), is characteristic of a process parameter, selected from a group comprising a time, a temperature, a pressure, a tool, a pretreatment of a tool, a type of application of a reagent, and a sequence of addition of reagents.

7. The method according to claim 5 wherein the second process data record (VD₂) and/or at least one of the other process data records (VD₃ ... VDₙ) each contain at least 3 process data values (VDWₓ), which are characteristic of a process parameter for implementing a second or further filler composition (FM₂ FM₃ ... FMₙ) and/or a second or further composite mass (KM₂ KM₃ ... KMₙ) to form a second or further cast workpiece (GWS₂ GWS₃ ... GWSₙ) and which were determined during the conversion of a second or further filler composition (FM₂ FM₃ ... FMₙ) and/or a second or further composite mass (KM₂ KM₃ ... KMₙ) into a second or further cast workpiece (GWS₂ GWS₃ ... GWSₙ).

8. The method according to claim 7, wherein at least one of the other process data records (VD₃ ... VDₙ) each contain at least 10 process data values (VDWₓ).

9. The method according to claim 6, wherein the second process data record (VD₂) and/or at least one of the other process data records (VD₃ ... VDₙ) each contain at least 3 process data values (VDWₓ), which are characteristic of a process parameter for implementing a second or further filler composition (FM₂ FM₃ ... FMₙ) and/or a second or further composite mass (KM₂ KM₃ ... KMₙ) to form a second or further cast workpiece (GWS₂ GWS₃ ... GWSₙ) and which were determined during the conversion of a second or further filler composition (FM₂ FM₃ ... FMₙ) and/or a second or further composite mass (KM₂ KM₃ ... KMₙ) into a second or further cast workpiece (GWS₂ GWS₃ ... GWSₙ).

10. The method according to claim 9, wherein at least one of the other process data records (VD₃ ... VDₙ) each contain at least 10 process data values (VDWₓ).

11. A computer-implemented method for generating a trained machine learning composite mass formulation model for determining at least one first property (E_{1[KM1]} E_{2[KM1]}…; E_{1[GWS1]}E_{2[GWS1]} ...) of a first filler composition (FM₁) and/or a first composite mass (KM₁) and/or a first cast workpiece (GWS₁) that can be produced therefrom based on the use of composition datasets (ZD₂ - ZDₙ), each of which is characteristic of a filler composition (FM₂ ...FMₙ), composite mass (KM₂ ... KMₙ) and/or a cast workpiece (GWS₂ ... GWSₙ),
wherein the composition dataset (ZD₂ - ZDₙ) comprises in each case at least one analysis variable (AG_{2-n[KM2-KMn]} AG_{2-n[GWS2-GWSn])} and a proportion value (N_{A2} … N_{An} N_{B2} … N_{Bn} N_{C2} … N_{Cn}11...) of a component (A₂ ... Aₙ B₂₋ₙ C₂₋ₙ ...) relating to the respective filler composition (FM₂ ... FMₙ) and/or the respective composite mass (KM₂ ... KMₙ) and/or the respective cast workpiece (GWS₂ ... GWSₙ),
wherein the analysis variable is characteristic of at least one property (E_{2-n[KM3-KMn]} E_{2-n[GWS3-GWSn]}) of the filler composition (FM₂ …FMₙ) and/or the composite mass (KM₂ ... KMₙ) and/or cast workpiece (GWS₂ ... GWSₙ), comprising:
providing a trainable composite mass formulation model for machine learning, the model comprising a set of trainable parameters and generating an analysis variable (AG_{1[FM1]}AG_{2[FM1]}…; AG_{1[KM1]} AG_{2[KM1]} …; AG_{1[GWS1]}AG_{2[GWS1]}11...) as an output variable, which is characteristic of at least one for a property (E_{1[FM1]}E_{2[FM1]} …;) of the first filler composition (FM₁), and/or for a property (E_{1[KM1]} E_{2[KM1]}…;) of the first composite mass (KM₁), and/or for a property (E_{1[GWS1]} E_{2[GWS1]} …) of the first cast workpiece (GWS₁) characteristic, based on a composition dataset (ZD₃ … ZDₙ) or data derived therefrom, as input variables, which is determined from at least one filler composition (FM₂ ... FMₙ) and/or a composite mass (KM₂ ... KMₙ) and/or a cast workpiece (GWS₂ … GWSₙ) ;
generating a training dataset comprising a plurality of recorded and/or determined composition datasets (ZD₂ … ZDₙ) for training filler compositions (FM₁11... FMₙ) and/or training composite masses (KM₂ ... KMₙ), and/or training cast workpieces (GWS₂ ... GWSₙ), which are used to generate the training datasets, as well as measurement data or analysis variables derived therefrom (AG_{2-n[KM2-KMn]} AG_{2-n[GWS2-GWSn]}), which are characteristic of at least one property (E_{1-n[FM2-n]}E_{2-n[FM2-n]}…; E_{1-n[KM2-n]}E_{2-n[KM2-n]}…; E_{1-n[GWS2-n]}E_{2-n[GWS2-n]} ...) of the respective training filler composition (FM₂ ... FMₙ) and/or the respective training composite mass (KM₂ ... KMₙ), and/or the training cast workpiece (GWS₂ ... GWSₙ); and
training the composite mass formulation model based on the training dataset.

12. A method comprising determining at least one analysis variable (AG_{1[FM1]} AG_{2[FM1]}…; AG_{1[KM1]}AG_{2[KM1]}…; AG_{1[GWS1]} AG_{2[GWS1]}…) characteristic of at least one property (E_{1[FM1]}E_{2[FM1]}…; E_{1[KM1],} E_{2[KM1]} …; E_{1[GWS1]} E_{2[GWS1]} …) of a first filler composition (FM₁), a first composite mass (KM₁), and/or a first cast workpiece (GWS₁) using the composite mass formulation model generated in claim 11.

13. A composite mass formulation determination device for determining at least one property (E_{1[FM1]}E_{2[FM1]}...; E_{1[KM1]} E_{2[KM1]} ...; E_{1[GWS1]} E_{2[GWS1]}...) of a first filler composition (FM₁) and/or a first composite mass (KM₁) and/or a first cast workpiece (GWS₁),
wherein the composite mass formulation determination device is suitable and designed to determine a second composition dataset (ZD₂) for a second filler composition (FM₂) and/or a second composite mass (KM₂) and/or a second cast workpiece (GWS₂) and at least one further composition dataset (ZD₃13... ZDₙ) for at least one further filler composition (FM₃ ... FMₙ) and/or a further composite mass (KM₃ ... KMₙ) and/or a further cast workpiece (GWS₃ ... GWSₙ), wherein the respective composition dataset (ZD₂ ZD₃ … ZDₙ) comprises at least one second or further analysis variable (AG_{2-n[FM3-FMn]} AG_{2-n[KM3-KMn]}AG_{2-n[GWS3-GWSn]})and at least one second or further proportion value (N_{A2} N_{A3} … N_{An}) of a second or further component (A₂ A₃ … Aₙ) and optionally a plurality of further proportion values (N_{B2} … N_{Bn} N_{C2} … N_{Cn} …) of second or further components (B₂₋ₙ C₂₋ₙ …),
wherein the analysis variable (AG_{2-n[FM3-FMn]} AG_{2-n[KM3-KMn]} AG_{2-n[GWS3-GWSn]})is characteristic of at least one property (E_{1-n[FM2-n]}E_{2-n[FM2-n]} …; E_{1-n[KM2-n]} E_{2-n[KM2-n]}...; E_{1-n[GWS2-n]} E_{2-n[GWS2-n]}...) of a second or further filler composition (FM₂₋ₙ) and/or a further composite mass (KM₂₋ₙ) and/or a second or further cast workpiece (GWS₂₋ₙ), and wherein the proportion value (N_{B2}13... N_{Bn} N_{C2} … N_{Cn} …) is characteristic of a quantity fraction of the second and/or further second or further components (A₂ A₃ … Aₙ) present in the respective filler composition (FM₂₋ₙ)and/or the respective composite mass (KM₂₋ₙ) and/or the respective cast workpiece (GWS₂₋ₙ),
wherein the composite mass formulation determination device is suitable and designed to determine, by means of a processor device, at least one value of a composition dataset (ZD₁), which contains data for the formulation of a first filler composition (FM₁) and/or a first composite mass (KM₁) for the production of a first cast workpiece (GWS₁), based on the second composition dataset (ZD₂) for the second filler composition (FM₂) and/or the second composite mass (KM₂) and/or the second cast workpiece (GWS₂) and the at least one further composition dataset (ZD₃ ... ZDₙ) for the at least one further filler composition (FM₃ ... FMₙ) and/or a further composite mass (KM₃ ... KMₙ) and/or the further cast workpiece (GWS₃ ... GWSₙ).
